(19) United States
(12) Patent Application Publication
(10) Pub. No.: US 2011/0257584 A1
(43) Pub. Date: Oct. 20, 2011

(54) METHODS AND DEVICES FOR INJECTION OF A SUBSTANCE INTO TISSUE

(75) Inventors: Gregory B. Altshuler, Lincoln, MA (US); Ilya Yaroslavsky, North Andover, MA (US); David Tabatadze, Worcester, MA (US); Valery V. Tuchin, Saratov (RU); Andrei Belikov, St. Petersburg (RU); Andrei Erofeev, North Andover, MA (US)

(73) Assignee: PALOMAR MEDICAL TECHNOLOGIES, INC., Burlington, MA (US)

(21) Appl. No.: 13/088,095

(22) Filed: Apr. 15, 2011

Related U.S. Application Data
(60) Provisional application No. 61/324,977, filed on Apr. 16, 2010.

(51) Int. Cl. A61M 37/00

(52) U.S. Cl. 604/20

(57) ABSTRACT

Substances are injected (e.g. laser injected) into skin tissue in order to change optical and/or mechanical properties of the tissue. Methods include ablating one or more micro-holes into tissue, pushing a substance into the one or more micro-holes with energy from the creation of the mirco-holes, with acoustic energy, and/or with laser energy. A container component is filled with the substance to be injected into the skin tissue.

### RELATED APPLICATION

This application claims the benefit of and priority to and is the non-provisional application of U.S. Ser. No. 61/324,977 filed Apr. 16, 2010, entitled “Methods and Devices for Optical Clearing of Skin Tissue.”

### BACKGROUND

The desirability of using optical clearing substances to alter the translucence, transparency, and/or opacity of skin tissue has been discussed. This disclosure incorporates by reference the disclosure of U.S. Ser. No. 12/206,426 entitled “Methods and Devices for Fractional Ablation of Tissue for Substance Delivery,” which discusses the use of clearing substances for altering the transparency of tissue. In accordance with one embodiment of the prior disclosure, in an in vitro experiment on pig skin, micro-holes were formed with a device at a wavelength of 2940 nm and clearing substances were introduced into the tissue through the micro-holes by simple diffusion. In vitro experiments indicated that molecules could penetrate into the tissue through drilled micro-holes by simple diffusion. The success in the in vitro experiments did not translate into results that would work as desired in vivo. For example, the level of penetration of the molecules did not provide the desired level of optical clearing. In addition, a lapse of time (e.g., multiple minutes and/or hours) was required for diffusion to occur. Attempts were made to drill micro-holes using a device at a wavelength range that includes 2940 nm and then employ water pressure to force (e.g., inject) a substance into the micro-holes. In vitro, the level of water pressure required to achieve a desired or a targeted level of molecule penetration was so high that it would not be tolerated by patients due to discomfort and/or pain.

### SUMMARY

Substances can be injected (e.g., laser injected) into tissue to change optical and/or mechanical properties of the tissue (e.g., skin tissue). Optical changes can include providing protection from UV-light (e.g., by laser injection of, for example, TiO₂ and/or Al₂O₃ and/or ZrO₂ into the skin). Optical changes can also include a cosmetic change of the visual appearance of the skin (e.g., a cosmetic improvement by injection of, for example, TiO₂ and/or Al₂O₃ and/or ZrO₂ into the skin). Optical changes can include optically clearing at least a portion of the skin (e.g., covering all or a portion of a tattoo, lightening or darkening the visual appearance of skin to make the skin appear to have a more even tone by injection of, for example, TiO₂ and/or Al₂O₃ and/or ZrO₂ into the skin). Suitable substances that may be laser injected into tissue to change optical and/or mechanical properties of the skin tissue include, by way of non-limiting example, TiO₂ (with, for example, a 100 nm particle size) and/or Al₂O₃ (with, for example, a 27 μm particle size) and/or ZrO₂ (with, for example, a 5 μm particle size) and/or hydrocortisone. In one embodiment, any of TiO₂ and/or Al₂O₃ and/or ZrO₂ can have a concentration of 5 to 500 mg/ml in a suspension such as, for example, PEG (polyethylene glycol). Any of a number of suitable biocompatible suspension mediums can be employed such as, for example, PEG, ethylene glycol, polypropylene glycol, and glycerol, for example.

Any of a number of substances, concentrations of substances, and/or particle size of substances may be suited to laser injection to provide a change to optical and/or mechanical properties of tissue.

Substances can change the mechanical properties of the tissue and can include changing the elasticity of tissue. Injecting fillers into tissue can be employed to alter the mechanical properties of the tissue. Tissue can be made, for example, more rigid, denser by using filler (e.g., a substance that has a higher density than the skin tissue). Materials that can be suited to change the mechanical properties of tissue include, for example, biologically compatible products such as collagen fillers (e.g., chemically modified collagen fillers that are cross-linked to enhance the “life” of the filler in use). Some suitable substances that can be employed to change the mechanical properties of the tissue can include, for example, Hyaluronic Acid (e.g., the brand name Juvederm), Calcium Hydroxylapatite (e.g., the brand name Radiesse), and PMMA (polymethylmethacrylate) (e.g., the brand name Artefill). Some of the previously disclosed substances employed to alter the optical appearance of the skin tissue can also impart mechanical changes to the skin tissue. For example, certain concentrations and/or particle size(s) of TiO₂ and/or Al₂O₃ and/or ZrO₂ can alter mechanical properties of the tissue.

In one aspect a method is provided for driving a substance into a subject's skin. The method includes placing a substance in contact with or in proximity to a portion of the skin and applying energy to the skin portion so as to generate a plurality of micro-holes in the skin and applying energy to at least a portion of the substance to generate pressure for forcing at least a portion of the substance into the micro-holes. In some embodiments, at least a portion of the substance changes its phase into a gaseous phase and/or at least a portion of the substance changes its phase into a liquid phase in response to the applied energy.

The substance can be disposed in a container having a surface adapted for contact with the skin. Optionally, the surface adapted for contact with the skin surface is frangible and perforates in response to the application of energy to a portion of the container. In some embodiments, the container is maintained in contact with the skin during the application of the energy to the skin and to the substance.

The substance that is driven into the subject's skin may include one or more of particles, molecules, molecular compounds, suspensions, gels, and/or liquids. The substance can alter the optical properties of skin. For example, the substance can optically clear at least a portion of the skin appearance (e.g., cover all or a portion of a tattoo). The substance can whiten, lighten and/or darken the visual appearance of a region of skin tissue (e.g., so that the skin tissue appears to have a more even tone). The substance can protect at least a portion of the skin from UV-light light (e.g., by laser injection of a UV-protectant such as TiO₂ into the skin).

In one embodiment, the substance disposed within the micro-holes changes over time such that the optics of the skin returns to the unaltered optical appearance. For example, the substance can degrade (e.g., over a period of time) such that it alters the optical properties of the region of skin tissue (e.g., to cover all or a portion of a tattoo) for a limited amount of time (e.g., until the substance degrades). This way, the optical appearance of skin tissue can be altered for a limited period of time (e.g., an otherwise desired tattoo can be covered for an occasion where the appearance of a tattoo may be undesirable).

In some embodiments, the plurality of micro-holes can have a depth at or above the dermal epidermal junction and the substance forced into the micro-holes are at a depth at or above the dermal epidermal junction. Disposing the substance into micro-holes at a depth at or above the dermal epidermal junction enables the substance to have a temporary effect. Without being bound by a single theory, by disposing the substance at or above the dermal epidermal junction it is expected that the substance will be “shedded” based upon the cycle of epidermal growth of skin and/or sloughing of skin. For example, disposing the substance at a depth at or above the dermal epidermal junction can enable temporary masking of a tattoo (e.g., for a special event or for a desired period of time). By disposing the substance at or above the dermal epidermal junction it is expected that the tattoo particles being masked will be revealed in time based upon the cycle of epidermal growth of skin and/or sloughing of skin.

In other embodiments, the plurality of micro-holes can have a depth below the dermal epidermal junction and the substance forced into the micro-holes can be at a depth below the dermal epidermal junction. Masking below the dermal epidermal junction can enable permanent or substantially permanent coverage of tattoo particles.

In one embodiment, the substance is disposed in a container and the container provides a seal with the skin when the container is in contact with the skin.

In another aspect, a method is provided for driving a substance into a subject's skin. The method includes placing a container housing in contact with a portion of the subject's skin. The container housing defines a compartment containing a substance, the container housing is configured to seal the compartment between the subject's skin and the container housing when the compartment is in contact with the skin. The method includes applying ablative energy through at least a portion of the container housing thereby ablating the skin portion (through the container component) and so as to generate a plurality of micro-holes. The pressure within the compartment increases due to generation of the plurality of micro-holes and the pressure increase can drive at least a portion of the substance into the micro-holes.

In some embodiments, at least a portion of the substance driven into said micro-holes is in a gaseous phase and/or is in the liquid phase. The container housing can have a frangible surface that is perforated by the application of ablative energy to the container housing.

In some embodiments, the container housing is maintained in contact with the skin throughout the application of the ablative energy to the skin and the substance.

The substance that is being disposed in the micro-holes can alter the optics of the tissue, for example, the substance can optically clear at least a portion of the skin appearance, can lighten at least a portion of the skin appearance, and/or can protect at least a portion of the skin from UV-light.

In some embodiments, the plurality of micro-holes have a depth at or above the dermal epidermal junction and the substance forced into the micro-holes can be at a depth at or above the dermal epidermal junction. In other embodiments, the plurality of micro-holes can have a depth below the dermal epidermal junction and the substance forced into the micro-holes can be at a depth below the dermal epidermal junction.

In another aspect, a container component is provided that can be used for driving a substance into tissue. The container component includes a compartment having a window and a wall. At least a portion of the window is optically transparent to laser energy and the window reflects at least a portion of the acoustic (or sonic) energy created when the laser energy is applied to tissue and prevents escape of the reflected acoustic (or sonic) energy from the within the compartment. At least a portion of the wall can form a seal with the tissue surface. In one embodiment, the window comprises sapphire. Suitable materials that can be employed to make all or a portion of the container component and are capable of retaining and reusing shock energy include, for example, plastic (e.g., hard plastic), quartz, and/or sapphire, used alone or in combination with one another or with other materials.

In some embodiments, the reflected acoustic (or sonic) energy is imparted to the substance thereby increasing the pressure within the compartment. The energy imparted to the substance can transform all or a portion of the substance into a gas and/or a liquid.

In one embodiment, the container further comprises an orifice through which a substance is inserted into the compartment.

### DETAILED DESCRIPTION OF THE FIGURES

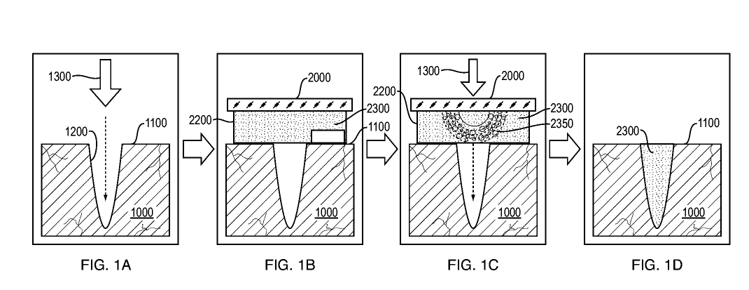

FIGS. 1A-1D shows a method of laser injection of a substance into skin tissue by forming one or more micro-holes in tissue using a laser having a wavelength suited to tissue ablation (FIG. 1A); positioning a container component having a substance within its internal chamber on the surface of the tissue (FIG. 1B); a device that delivers energy delivers to the container component energy suitable to generate pressure for forcing at least a portion of the substance within the internal chamber into the one or more micro-hole (FIG. 1C); the increase in pressure forced at least some of the substance into the one or more micro-hole (FIG. 1D).

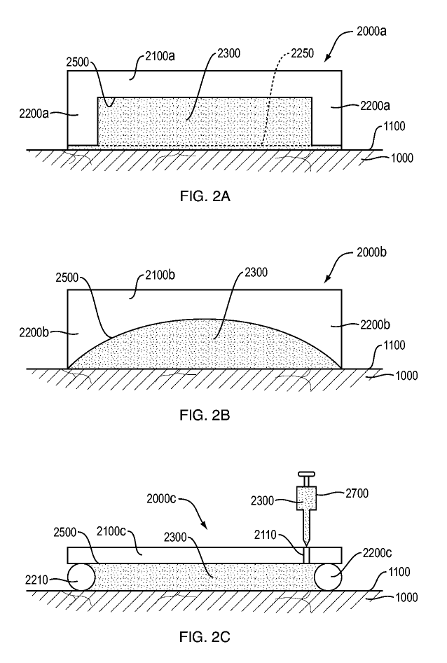

FIG. 2A shows a cross section of a container component as described in association with FIG. 1B. The container component has a window and sidewalls that are at an angle substantially perpendicular to the window. The container component optionally has a lid that enables the container component to be pre-filled with the substance.

FIG. 2B shows a cross section of a container component as described in association with FIG. 1B. The container component has a window and sidewalls. The window and the sidewalls can be a single unit.

FIG. 2C shows a cross section of a container component as described in association with FIG. 1B. The container component has a window and sidewalls. The sidewalls can be made from an O-ring that is disposed permanently or removably on the window. The container component optionally has a lid that enables the container component to be pre-filled with the substance.

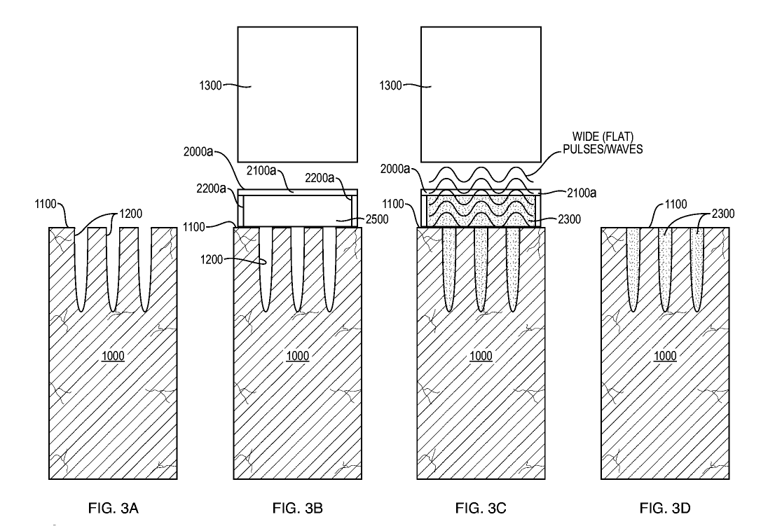

FIGS. 3A-3D shows a method of laser injection of a substance skin tissue by forming a plurality of micro-holes in tissue using a laser having a wavelength suited to tissue ablation (FIG. 3A); positioning a container component having a substance within its internal chamber on the surface of the tissue (FIG. 3B); a device that generates energy (such as a laser or an ultrasound device) delivers to the container component energy suitable to generate pressure for forcing at least a portion of the substance within the internal chamber into the micro-holes (FIG. 3C); the increase in pressure forces at least some of the substance into the micro-holes (FIG. 3D).

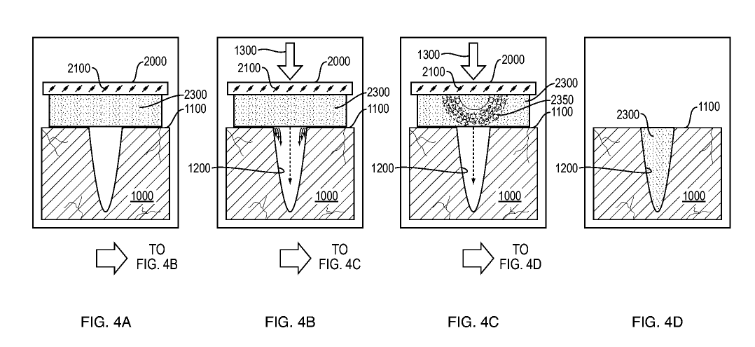

FIGS. 4A-4D shows a method of laser injection of a substance into tissue by positioning a container component having a substance within its internal chamber on the surface of the tissue, the container component having a window that is at least partially transparent to energy transmission (FIG. 4A); delivering a pulse of ablative energy through the window, through the substance, and through at least a portion of the tissue, thereby forming at least one micro-hole in the tissue (FIG. 4B); pressure forces at least a portion of the substance within the internal chamber into the at least one micro-hole (FIG. 4C); the tissue having at least one micro-hole is at least partially filled with the substance after the container component is removed from the skin surface (FIG. 4D).

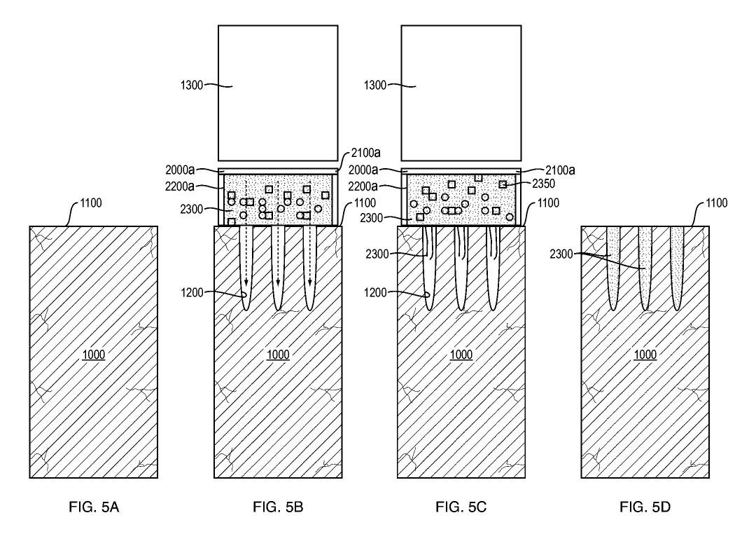

FIGS. 5A-5D shows a method of laser injection of a substance into tissue by providing intact tissue (FIG. 5A); placing a container component adjacent a surface of intact tissue, the container component includes a window and has an internal chamber containing a substance, delivering a plurality of ablative microbeams through the window of the container component, to form a plurality of micro-holes, the tissue ablated during the formation of the micro-holes increases the pressure in the internal chamber of the container component that holds the substance (FIG. 5B); the expansion in the volume of the internal chamber caused at least in part by the ablated skin tissue material causes at least a portion of the substance to be displaced from within the internal chamber of the container component to fill at least a portion of the plurality of micro-holes (FIG. 5C); the tissue having a plurality of micro-holes is at least partially filled with the substance after the container component is removed from the skin surface (FIG. 5D).

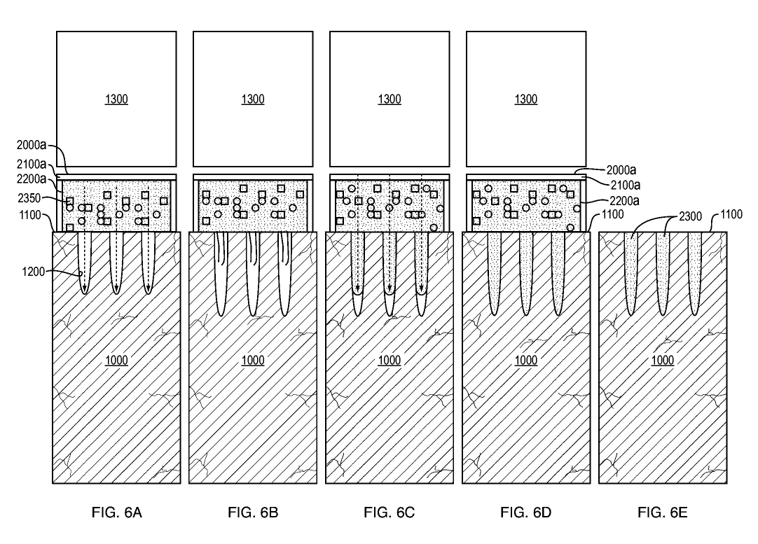

FIGS. 6A-6E shows a method of laser injection of a substance into tissue by placing a container component adjacent a surface of intact tissue, the container component includes a window and has an internal chamber containing a substance, delivering a plurality of ablative microbeams through the window of the container component, through the substance disposed in the internal chamber and through the skin tissue to form a plurality of micro-holes, the tissue ablated during the formation of the micro-holes increases the pressure in the internal chamber of the container component that holds the substance (FIG. 6A); the expansion in the volume of the internal chamber caused at least in part by the ablated skin tissue material causes at least a portion of the substance to be displaced (e.g., pushed) from within the internal chamber of the container component to fill at least a portion of the plurality of micro-holes (FIG. 6B); an energy generating source delivers energy through the window of the container at an energy level less than is required to ablate skin tissue, but sufficient to push (e.g., force) at least a portion of the substance into the one or more of the plurality of micro-holes (e.g., the substance may be pushed due to the conversion of at least a portion of the substance into gas) (FIGS. 6C and 6D); the tissue having a plurality of micro-holes is at least partially filled with the substance after the container component is removed from the skin surface (FIG. 6E).

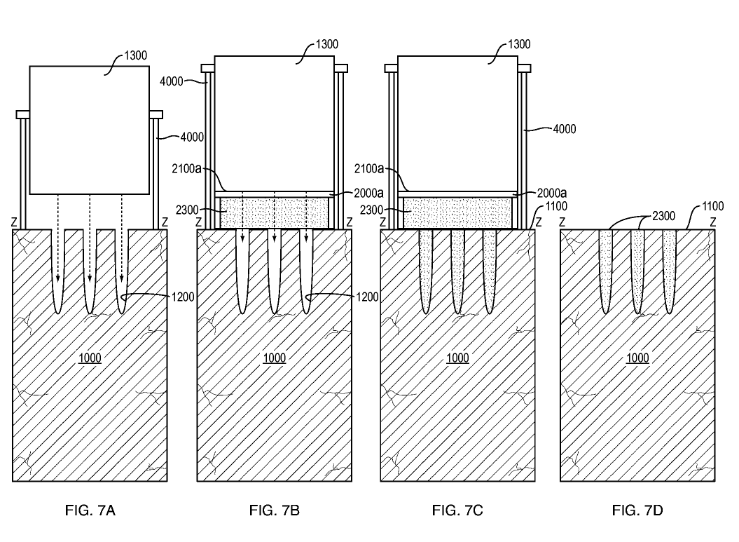

FIGS. 7A-7D shows a method of laser injection of a substance into skin tissue using an aligner with a container component. An aligner is positioned adjacent the surface of the tissue to be treated and an energy source is positioned within the aligner and delivers a plurality of ablative microbeams through the surface of the skin tissue (FIG. 7A); a container component having an internal chamber filled with a substance is placed on the surface of the skin tissue in at least partial alignment with the aligner, the energy source delivers a plurality of micro beams through the window of the container component thereby increasing the pressure within the internal chamber of the container component (FIG. 7B); the increased pressure within the internal chamber pushes and/or forces at least a portion of the substance into one or more of the plurality of micro-holes (FIG. 7C); the tissue having a plurality of micro-holes is at least partially filled with the substance after the container component and/or the aligner is removed from the skin surface (FIG. 7D).

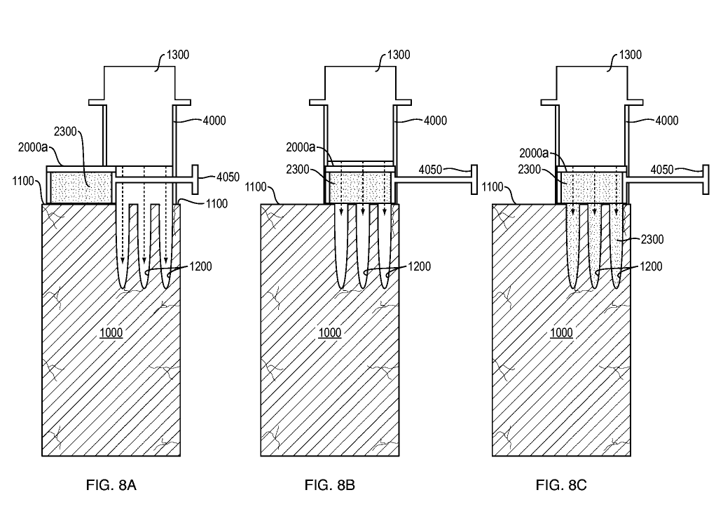

FIGS. 8A-8C shows a method of laser injection of a substance into skin tissue using an aligner with a container component. An aligner is positioned adjacent the surface of the tissue to be treated and an energy source is positioned within the aligner and delivers a plurality of ablative microbeams through the surface of the skin tissue thereby forming a plurality of micro-holes (FIG. 8A); a handle is used to pull the container component over the surface of the skin tissue into the region of the skin tissue where the microbeams were previously delivered by the laser, the energy source delivers a wavelength suitable to increase the pressure of the substance held within the container component (FIG. 8B); the increase in pressure in the internal chamber caused by the energy delivered to the substance held within the container component pushes at least a portion of the substance into one or more of the plurality of micro-holes.

### DETAILED DESCRIPTION

In one aspect, a substance suitable for changing the visual appearance of tissue (e.g., suitable for “optical clearing” of tissue) is “laser injected” into tissue. Such substances can include, for example, particles, molecular compounds, suspensions, gels, and/or liquids. Substances suitable for optical clearing can include, for example, ZnO₂ (e.g., Zirconium (IV) oxide powder <5 micron, 99% metals basis available form Sigma-Aldrich Chemie GmbH) and glycerin. Optical clearing can include an at least partial change (e.g., improvement) of the appearance of the tissue.

Laser injection delivers the substance into the tissue to a desired (e.g., targeted) depth. Suitable depths can include the depth from the surface of the skin tissue of from about 0 microns to about 20 microns (e.g., the depth of the stratum corneum) and from about 0 mm to about 10 mm (e.g., the depth of muscle). Laser injection can be employed, for example, to improve the appearance of a tattoo by covering all or a portion of the tattoo with the substance. In one embodiment, laser injection employs a laser having a wavelength that includes 2940 nm (e.g., the erbium laser) to improve tattoo removal by a) at least partially clearing the skin of the appearance of the tattoo and b) delivering particle(s) to the tissue.

In one embodiment, clearing skin with laser injection involves drilling “micro-holes” in the skin to deliver a substance that creates a cleared appearance in the skin. As will be appreciated by a person skilled in the art, “micro-holes” can have various dimensions. By way of non-limiting example, the micro-holes can have a depth extending about 0 microns from the skin surface, or to about 50 microns, about 100 microns, about 200 microns, about 300 microns, about 500 microns, about 1000 microns, about 2000 microns, about 5000 microns, or to about 10000 microns below the skin surface. The micro-holes can also have cross-sections of various widths. By way of non-limiting example, the micro-holes can have a maximum width at the skin surface of about 1 micron, about 10 microns, about 20 microns, about 50 microns, about 100 microns, about 200 microns, about 400 microns, about 1000 microns, about 1200 microns, or about 2000 microns. The width of the micro-holes can be substantially uniform or vary along their depth.

To create a cleared appearance, for example, the skin tissue ablated to form micro-holes in the tissue (e.g., fluid, water and extracellular material) can be replaced with high refractive index material(s) such as glycerin, which makes skin more transparent by decreasing scattering. In one embodiment, a substance (e.g., particles) is delivered to the micro-holes and replaces the fluid in the micro-holes prior to micro-hole formation to mask the appearance of all or a portion of a tattoo due to effective light scattering. Optionally, the substance (e.g., at least some of the delivered particles) is colored to match the subject's skin tone in the region being covered to mask a tattoo and/or to blend into the subject's surrounding skin.

FIGS. 1A-1D and 2A-2C show a method in which skin tissue 1000 is treated by laser injection. In accordance with this method, a micro-hole 1200 is formed in the tissue 1000 with a laser 1300 delivering a wavelength range suitable for tissue 1000 ablation. For example, the wavelength range can include one or more wavelength(s) in a range from about 1.8 microns to about 11 microns, from about 1800 nm to about 3500 nm, or from about 180 nm to about 350 nm. In one embodiment, referring to FIG. 1A, a wavelength range including 2940 nm can be employed to form, “drill” or “blow” one or more micro-holes 1200 into the tissue 1000. The energy density of the applied energy can range from about 0.1 J/cm² to about 500 J/cm².

FIGS. 1B and 2A-2C each show a cross section of a container component 2000, 2000A, 2000B, and 2000C positioned on the surface 1100 of the skin tissue 1000 containing the micro-hole 1200 (pre-ablated in FIG. 1A). The container component 2000 contains a substance 2300 within its internal chamber 2500. The substance can be, for example, in liquid and/or in gel and/or in solid form. Suitable substances can include a suspension of inert and/or biologically active particles in one or more solvents, solutions of one or more types of molecules dissolved in one or more solvents, a mixture of several solvents, a gel such as a gel containing a matrix of inert and/or biologically active particles and/or one or more types of molecules.

FIG. 2A shows a cross section of a container component 2000A that may be positioned on the surface 1100 of the skin tissue 1000. The container component 2000A has a window 2100A and a side wall labeled 2200A. All or a portion of the window 2100A is optically transmissive to one or more laser wavelength(s) that are pulsed therethrough. The side wall 2200A shown in FIG. 2A can be at an angle substantially perpendicular to the window 2100A. The window 2100A and the side wall 2200A create an internal chamber 2500. The internal chamber 2500 holds the substance 2300. In some embodiments, the container component 2000A optionally has a lid 2250 that holds the substance 2300 in the internal chamber 2500 of the container component 2000A. The lid 2250 enables the internal chamber 2500 to be pre-filled with the substance 2300. The lid 2250 can be removable such that it is peeled from a surface of the side wall 2200A prior to placing the substance 2300 and the side wall 2200A of the container component 2000A against the surface 1100 of the skin tissue 1000. In some embodiments, the lid 2250 can be made from a frangible material that breaks when a pulse of a laser 1300 or other energy source is applied through the window 2100A, the substance 2300, and the lid 2250. In one embodiment, when the container compartment 2000A is placed on the surface 1100 of the skin tissue 1000, the side wall 2200A creates a seal with the surface 1100 of the skin tissue 1000.

FIG. 2B shows a cross section of a container component 2000B that can be positioned on the surface 1100 of the skin tissue 1000. The container component 2000B has a window 2100B and a side wall labeled 2200B. The window 2100B and the side walls 2200B can be a single unit. All or a portion of the window 2100B and/or the side walls 2200B are optically transmissive to one or more laser wavelength(s) that are pulsed therethrough. The window 2100B and the side wall 2200B can be and/or can function as described above with reference to FIG. 2A.

FIG. 2C shows a cross section of a container component 2000C that may be positioned on the surface 1100 of the skin tissue 1000. The container component 2000C has a window 2100C and a side wall 2200C. The side wall 2200C can be an O-ring 2210 that is disposed (e.g., permanently or removably) on the window 2100C. All or a portion of the window 2100C and/or the side wall 2200C can be optically transmissive to one or more laser wavelength(s) that are pulsed through the window 2100C. The window 2100C and the side wall 2200C create an internal chamber 2500. The internal chamber 2500 holds the substance 2300. The container component 2000C can optionally have a lid (not shown) that holds the substance 2300 in the internal chamber 2500 of the container component 2000C, as described above with reference to FIG. 2A.

Referring still to FIG. 2C, optionally, the container component 2000C has an orifice 2110. The substance 2300 can be inserted into internal chamber 2500 (e.g., the compartment) through the orifice 2110. In one embodiment, the substance 2300 is pushed into the internal chamber 2500 via an external device 2700. Suitable external devices can include, for example, a syringe filled with the substance 2300 and with a plunger that pushes the substance 2300 into the internal chamber 2300 through, for example, the orifice 2110. The external device 2700 can be any means suited to introducing the substance 2300 to the internal chamber 2500 through the orifice 2110. In some embodiments, the external device 2700 delivers the substance 2300 to the internal chamber 2500 through the window 2100. In other embodiments, the external device 2700 delivers the substance 2300 to the internal chamber 2500 through the side wall 2200.

In one embodiment, the external device 2700 inserts the substance 2300 into the internal chamber 2500 prior to laser injection of the substance 2300 contained in the internal chamber 2500 into the surface 1100 of the skin tissue 1000. The container component 2000 can be moved to another skin tissue 1000 region and the external device 2700 can insert additional substance 2300 into the internal chamber 2500 prior to laser injection of the substance 2300 into another region of skin tissue 1000. In this way, the external device 2700 can be employed to refill the internal chamber 2500 with the substance 2300 between each laser injection “cycle.”

Optionally, the container component 2000 can be refilled with substance 2300 one or more times using the external device 2700 while the container component remains in one region of skin tissue. This way, the desired quantity of substance 2300 can be inserted into the micro-holes.

Referring now to FIGS. 1B and 1C and 2A-2C, when the container component 2000 is placed on the surface 1100 of the skin tissue 1000 the side wall 2200 can form a seal with the tissue surface 1100. FIG. 1C shows that the laser 1300 delivers a wavelength range suitable to generate pressure suited to forcing at least a portion of the substance into the one or more micro-holes. Alternatively, the device that delivers energy can be, instead of a laser, a lamp (generating a band of wavelengths) or an ultrasound device (delivering ultrasound or shock waves) that delivers energy through the window of the container component 2000 to reach the substance 2300.

For example, the device can be a laser 1300, the laser 1300 delivers energy at a wavelength range that is suitable to convert at least a portion of the substance 2300 into the form of gas 2350 and/or a liquid. For example, the range can include wavelength(s) from about 1.8 microns to about 11 microns, from about 1800 nm to about 3500 nm, or from about 180 nm to about 350 nm. In one embodiment, a single wavelength pulse suitable to convert the substance 2300 to a gas 2350 is employed. Suitable energy levels that may be employed can be high enough to convert at least a portion of the substance 2300 from a liquid and/or a gel and/or a solid into the form of a gas 2350 (e.g., ablate the substance 2300) but lower than the energy level required to ablate the tissue 1000. Some suitable energy levels can convert at least a portion of the substance 2300 from a gel and/or a solid form to a liquid form. For example, the energy density can range from about 1% to about 90% of the energy density used to ablate the tissue 1000. When all or a portion of the substance 2300 in the container component 2000 is converted to gas and/or a liquid the pressure within the internal chamber 2500 of the container component 2000 increases as the substance 2300 expands during its conversion to a gaseous form.

Referring now to FIG. 1D, the increase in pressure within the internal chamber 2500 forces (e.g., pushes) at least some of the substance 2300 into the micro-hole 1200, the increase in pressure within the internal chamber 2500 can be caused by the gas 2350. The seal held between the side wall of the container component 2000 and the surface 1100 of the skin tissue 1000 aids in ensuring that the substance 2300 is pushed into the micro-hole 1200. In some embodiments, the seal between the side wall of the container component 2000 and the surface 1100 of the skin tissue 1000 is imperfect such that at least a portion of the seal is between the side wall of the container component and the skin surface is breached.

The depth of substance 2300 delivery can depend at least in part on the depth of the micro-hole 1200 from the surface 1100 of the skin tissue 1000. In some embodiments, the micro-hole 1200 has a depth from the surface 1100 of the skin tissue 1000 of from about 0 microns to about 20 microns (e.g., the depth of the stratum corneum). In another embodiment, the micro-hole 1200 has a depth from the surface 1100 of the skin tissue 1000 of from about 0 mm to about 10 mm (e.g., the depth of muscle).

FIGS. 3A-3D show a method in which skin tissue 1000 is treated by laser injection. In FIG. 3A a plurality of ablated micro-holes 1200 are formed in the tissue 1000 by, for example, a laser having a wavelength range suitable for ablation. For example, the range can include one or more wavelength(s) in a range from about 1.8 microns to about 11 microns, from about 1800 nm to about 3500 nm, or from about 180 nm to about 350 nm. For example, the laser can have a wavelength of about 2940 nm. In FIG. 3B, a container component 2000A is positioned on the surface 1100 of the skin tissue 1000 containing the plurality of ablated micro-holes 1200. Optionally, the plurality of micro-holes 1200 are created by methods other than ablation (e.g., needles) prior to placing the container component 2000A on the surface 1100 of the skin tissue 1000 containing the plurality of micro-holes 1200. The container component 2000A contains a substance 2300 within its internal chamber 2500. FIG. 3C shows that a device that generates energy such as a laser 1300 (generating pulses) or a lamp (generating a band of wavelengths) or an ultrasound device (delivering ultrasound or shock waves) delivers energy through the window 2100A of the container component 2000A to reach the substance 2300. The energy that is transmitted through the window 2100A is suitable to generate pressure for forcing at least a portion of the substance into the micro-holes 1200. The energy transmitted through the window 2100A can convert at least a portion of the substance 2300 from a solid and/or a liquid and/or a gel into the form of a gas 2350 or a liquid. Suitable energy levels that can be employed can be high enough to convert at least a portion of the substance 2300 from a solid and/or liquid and/or a gel into the form of a gas 2350 or a liquid (e.g., energy suited to ablate the substance 2300) but lower than the energy level required to ablate the tissue 1000. In one embodiment, a laser 1300 provides a single wavelength pulse suitable to convert the substance 2300 to a gas 2350 or a liquid. When all or a portion of the substance 2300 in the container component 2000A is converted to gas and/or a liquid the pressure within the internal chamber 2500 of the container component 2000A increases as the substance 2300 expands during its conversion to a gaseous and/or to a liquid form. The increase in pressure within the internal chamber 2500 forces at least some of the substance 2300 into the plurality of micro-holes 1200 in the tissue 1000. In this step, the delivery of acoustic and/or light based energy (e.g., from a laser or from a lamp) to the substance 2300 causes at least a portion of the substance 2300 to be delivered into the plurality of micro-holes 1200 when the substance having been converted into gaseous and/or liquid form pushes into the available space (e.g., the void) within the micro-holes 1200.

In some embodiments, the window 2100A and/or walls 2200A of the container component 2000A can be made from a material having the capacity to hold at least a portion of the shock energy within the container component 2000A (e.g., within its internal chamber 2500). Where the container component 2000A is made of materials that can at least partially retain the shock energy created by, for example, the laser and/or other energy source, the shock energy can be reused to aid in forcing and/or pushing the substance into the one or more micro-holes. Suitable materials that can be employed to make all or a portion of the container component capable of retaining and reusing shock energy include, for example, plastic (e.g., hard plastic), quartz, and/or sapphire.

In some embodiments, the longer the ultrasound energy is applied to the container component the deeper the penetration of the substance into the micro-holes. Suitable ultrasound settings include 1.5 W/cm² at 1 MHz, with CW (running continuously) for exposure times of ultrasound to the container component that range from 1 minute, 5 minutes, 10 minutes, or 30 minutes, for example.

The depth of substance 2300 delivery can depend at least in part on the depth of the micro-holes 1200 from the surface 1100 of the skin tissue 1000. In some embodiments, one or more of the micro-holes 1200 has a depth from the surface 1100 of the skin tissue 1000 of from about 0 microns to about 20 microns (e.g., the depth of the stratum corneum). In another embodiment, one or more of the micro-holes 1200 has a depth from the surface 1100 of the skin tissue 1000 of from about 0 mm to about 10 mm (e.g., the depth of muscle). Optionally, in a single treatment area having a plurality of micro-holes, adjacent micro-holes can have differing depths from the surface 1100 of the skin tissue 1000.

The container component 2000A can be removed from the surface 1100 of the skin tissue 1000 and FIG. 3D shows that the substance 2300 is trapped within the skin tissue 1000. In some embodiments, some substance 2300 in liquid or gel form that is left on the surface 1100 of the skin tissue 1000 can be wiped off of the skin tissue surface 1100 after the container component 2000A is removed. In some embodiments, after the container component 2000A is removed at least some of the substance 2300 (in liquid and/or in gel form) remains in the internal chamber 2500 of the container component 2000A.

In one embodiment, pig skin bearing tattoos was treated in vivo with a Lux 2940 handpiece having a groove optic with four pulses applied with each pulse having an energy level of 1 J to two regions of skin tissue. To the first skin region, TiO₂ was inserted to mask the tattoo and to the second skin region, Al₂O₃ was inserted to mask the tattoo. More specifically, TiO₂ (100 nm particle size) particles (at a concentration of 0.08 g/ml in PEG-300) were delivered to the first skin region via ultrasound at 1.5 W/cm² and at 1 MHz with CW (e.g., running continuously) for 5 minutes. Next, Al₂O₃ (27 μm particle size) particles (at a concentration of 0.08 g/ml in PEG-300) were delivered to the second skin region via ultrasound at 1.5 W/cm² and at 1 MHz with CW (e.g., running continuously) for 5 minutes. Biopsy's taken after one month of in vivo treatment revealed the presence of TiO₂ and Al₂O₃ remaining in the pig skin.

FIGS. 4A-4D show a method in which skin tissue 1000 is treated by laser injection of a substance into the skin tissue 1000 by employing an energy emitting device and a container component 2000. In accordance with this method, referring to FIG. 4A, a container component 2000 is placed adjacent a surface 1100 of skin tissue 1000. The container component 2000 includes a window 2100 and an internal chamber. In one embodiment, the internal chamber contains a substance 2300 suitable for optical clearing via laser injection.

Referring now to FIG. 4B, an energy generating source such as a laser 1300 delivers a pulse of energy through the window 2100 of the container component 2000. All or a portion of the window 2100 is transparent to the energy transmission delivered therethrough. In one embodiment, the laser 1300 delivers a pulse of ablative energy through the window 2100, through the substance 2300 and through the skin tissue 1000, thereby forming a micro-hole 1200 in the skin tissue 1000. For example, the pulse of ablative energy delivered through the window 2100 can ablate at least a portion of the substance 2300 and turn it into the form of a gas and/or a liquid and form the micro-hole 1200 in the skin tissue 1000 by ablation. Forming the micro-hole 1200 in the skin tissue 1000 creates pressure that forces (e.g., pushes) at least a portion of the substance 2300 (e.g., in gas form 2350) into the newly formed micro-hole 1200.

In another embodiment, referring to FIG. 4B in a first phase an energy generating source such as a laser 1300 delivers a pulse of ablative energy through the window 2100 transparent to the energy transmission delivered therethrough the substance 2300 and through the skin tissue 1000, thereby forming a micro-hole 1200 in the skin tissue 1000. For example, the energy density of the energy delivered to the skin can range from about 0.1 J/cm² to about 500 J/cm². Referring now to FIG. 4C, in a second phase an energy generating source such as a laser 1300 (e.g., a laser that is the same as or is different than the laser 1300 described in relation to FIG. 4B, or a lamp (generating a band of wavelengths) or an ultrasound device (delivering ultrasound or shock waves) is then employed to deliver a second pulse of energy through the window 2100 transparent to the energy transmission at an energy level that is less than the energy required to ablate the skin tissue 1000 but that can be sufficient to convert at least a portion the substance 2300 into a gas 2350 and/or a liquid. For example, the second pulse decreases the power or energy density such that the second pulse is below the threshold for skin ablation but is above the threshold for ablation of the substance. For example, the second pulse can have an energy density in the range of about 1% to about 90% of the energy density of the first pulse. The second pulse of energy pushes (e.g., forces) at least a portion of the substance 2300 into the micro-hole 1200. In one embodiment, the first pulse of energy ablates the micro-hole 1200 in the skin tissue 1000 and the second pulse of energy ablates the substance 2300 (but does not ablate the skin tissue 1000), thereby enabling the substance to be pushed into the micro-hole 1200. In some embodiments, the first pulse of energy that ablates the micro-hole 1200 is created by one laser and the second pulse of energy that ablates the substance 2300 is created by a source (e.g., laser, a lamp, an acoustic energy source) that is different from the laser that creates the first pulse. In other embodiments, the same laser is employed to deliver the first pulse and the second pulse, however, the laser employs one wavelength range to deliver the first pulse and another wavelength range to deliver the second pulse. For example, each of these wavelength ranges can include one or more wavelength(s) in a range from about 1.8 microns to about 11 microns, from about 1800 nm to about 3500 nm, or from about 180 nm to about 350 nm.

Once all or a portion of substance 2300, enclosed between the internal chamber 2500 of the container component 2000 and the surface 1100 of the skin tissue 1000, is turned from its original solid and/or liquid and/or gel state to gas, a high pressure system is created by the expansion of at least a portion of the substance 2300 to the gas 2350 and/or liquid form within the region between the chamber 2500 and the surface 1100 of the tissue 1000. An increase in pressure in the internal chamber 2500 environment forces at least a portion of the substance 2300 into the micro-hole 1200 created by the first pulse of energy. Without being bound to any single theory, it is believed that forming the micro-hole 1200 with the first pulse creates a pressure upon forming the micro-hole. The pressure created by the first pulse that forms the hole may force and/or push at least some of the liquid and/or gas into the micro-hole 1200.

FIG. 4D depicts the tissue 1000 having a micro-hole 1200 that is at least partially filled with the substance 2300 after the container component is removed from the surface 1100 of the skin tissue 1000. The depth of substance 2300 delivery can depend at least in part on the depth of the micro-hole 1200 from the surface 1100 of the skin tissue 1000. In some embodiments, the micro-hole 1200 has a depth from the surface 1100 of the skin tissue 1000 of about the stratum corneum (e.g., from about 0 microns to about 20 microns below the surface 1100 of the skin tissue 1000). In other embodiments, the micro-hole 1200 has a depth from the surface 1100 of the skin tissue 1000 of about the muscle (e.g., from about 0 mm to about 10 mm below the surface 1100 of the skin tissue 1000).

In one embodiment, the Palomar StarLux 500 with the Lux2940 hand piece using the “Groove” optic and the Lux2940 hand piece having a fractional tip with a pitch of 500 μm (750 μm on the skin) are employed to test the laser injection technique described in association with FIGS. 4A-4D. The StarLux 500 base unit is operated at parameters to provide energy per micro beam of 58 mJ at a pulse direction of about 135 μs. Relatively deep penetration of the substance can be realized with laser injection of the substance into the micro-holes. The particles can be delivered into the epidermis, into the dermis, and/or into the epidermis and the dermis. Laser injection reduces delivery time to less than 1 minute (compared to possibly greater than 10 minutes using ultrasound assisted delivery). Laser injection shows potential as a rapid and minimally-invasive transcutaneous delivery technique. Particles may stay within the skin in vivo for at least several weeks.

FIGS. 5A-5D shows a method in which skin tissue 1000 is treated by laser injection of a substance 2300 into the skin tissue 1000 by employing an energy emitting device 1300 and a container component 2000A. In accordance with this method, referring to FIGS. 5A and 5B, a container component 2000A is placed adjacent a surface 1100 of intact skin tissue 1000. The container component 2000A includes a window 2100A and an internal chamber containing a substance 2300 (e.g., a substance suitable for optical clearing). The substance 2300 can totally or substantially fill the chamber of the container component 2000A. The container component 2000A (e.g., the side of the container component 2000A that contacts the skin surface) retains contact with the surface 1100.

Referring now to FIG. 5B, an energy generating source such as a laser 1300 delivers a plurality of microbeams through the window 2100A of the container component 2000A. All or a portion of the window 2100A is transparent to the energy transmission delivered therethrough. The window 2100A can be made from, for example, sapphire or other material that is capable of holding in and reusing the shock energy delivered to the container component 2000A. In one embodiment, the laser 1300 delivers a plurality of microbeams of ablative energy through the window 2100A, through the substance 2300, and through the skin tissue 1000 thereby forming a plurality of ablated micro-holes 1200 in the skin tissue 1000 (e.g., a plurality of micro-holes 1200 that are complementary to the plurality of microbeams of ablative energy). During the ablation of the skin tissue 1000, the fluid in the ablated skin tissue boils and/or thermally evaporates such that upon ablation the intact skin becomes gas and micro particles. Each of the portions of ablated skin tissue that become gas and micro particles during the formation of micro-holes 1200 expand from an original volume to from about 10 times, about 20 times, about 50 times and/or about 70 times their original volume. Thus, forming the micro-holes 1200 in the skin tissue 1000 increases the pressure in the container component 2000A, specifically, the pressure in the internal chamber of the container component 2000A that holds the substance 2300 is increased by the expansion in volume of the skin tissue material that is ablated during the formation of the micro-holes 1200. Referring also to FIG. 5C, the expansion in the volume of the skin tissue material causes at least a portion of the substance 2300 to be displaced from within the internal chamber of the container component 2000A such that at least a portion of the substance 2300 moves into one or more of the plurality of micro-holes 1200 by, for example, displacement. Thus, the increase in pressure within the internal chamber of the container component 2000A caused by ablation of the micro-holes causes the substance 2300 to fill all or a portion of one or more of the plurality of micro-holes 1200. The increase in pressure within the container component forces or pushes at least a portion of the substance 2300 into one or more of the plurality of micro-holes 1200.

FIG. 5D depicts the tissue 1000 having a plurality of micro-holes 1200 that is at least partially filled with the substance 2300 after the container component is removed from the surface 1100 of the skin tissue 1000. The depth of substance 2300 delivery will depend at least in part on the depth of the plurality of micro-holes 1200 from the surface 1100 of the skin tissue 1000. In some embodiments, one or more of the micro-holes 1200 has a depth from the surface 1100 of the skin tissue 1000 of about the stratum corneum (e.g., from about 0 microns to about 20 microns below the surface 1100 of the skin tissue 1000). In other embodiments, the plurality of micro-holes 1200 have a depth from the surface 1100 of the skin tissue 1000 of about the muscle (e.g., from about 0 mm to about 10 mm below the surface 1100 of the skin tissue 1000).

Optionally, the delivery of the energy through the window 2100A shown in FIG. 5B to ablate the skin tissue 1000 additionally ablates at least a portion of the substance 2300. For example, referring to FIG. 5B, the pulse of ablative energy delivered through the window 2100A ablates at least a portion of the substance 2300 and turns it into the form of a gas and forms the plurality of micro-holes 1200 in the skin tissue 1000 by ablation. Thus, the transition of the substance 2300 into a gas form contributes to the increase in pressure in the container component 2000A and contributes to the displacement and/or pushing and/or forcing of at least a portion of the substance 2300 into one or more of the plurality of newly formed micro-holes 1200.

Referring now to FIGS. 6A-6E, treating tissue with laser injection can occur in multiple phases. Referring to FIG. 6A a container component 2000A is placed on the surface 1100 of intact skin tissue 1000. In a first phase, shown in FIG. 6A, an energy generating source such as a laser 1300 delivers a plurality of micro-beams energy, e.g., ablative energy, through the window 2100A of the container component 2000A, through the substance 2300 disposed in the internal chamber of the container component 2000A and through the skin tissue 1000 thereby forming a plurality of micro-holes 1200 in the skin tissue 1000. Ablating the plurality of micro-holes 1200 causes at least the fluid in the ablated tissue to turn into a gas and/or causes micro particles of ablated skin tissue 1000 to fill at least a portion of the container component 2000A. The gas and/or micro particles created by the ablated skin tissue takes up an increased volume compared to the volume of the previously intact skin tissue thereby increasing pressure in the internal chamber of the container component 2000A. FIG. 6B shows that forming the plurality of micro-holes 1200 in FIG. 6A increases the pressure in the container component 2000A due, at least in part, to the expansion in volume of the ablated tissue material within the container component 2000A. The increased pressure pushes (e.g., forces) at least a portion of the substance 2300 into one or more of the plurality of micro-holes 1200. The pressure generated by the first phase of ablative energy delivered to form the plurality of micro-holes 1200 may not generate enough pressure in the container component 2000A to fill one or more of the micro-holes 1200 with the desired quantity of the substance 2300. Referring now to FIG. 6C, in a second phase, an energy generating source such as a laser 1300 (e.g., a laser that is the same as or is different than the laser 1300 described in relation to FIG. 6B, or a lamp (generating a band of wavelengths) or an ultrasound device (delivering ultrasound or shock waves)) is then employed to deliver energy (e.g., a plurality of micro-beams) through the window 2100A at an energy level that is less than the energy required to ablate the skin tissue 1000 but that is sufficient to convert at least a portion of the substance 2300 into gas 2350 (e.g., the energy delivered can generate more gas 2350 than was generated in the first phase of energy delivery whereby micro-holes 1200 were created in the skin tissue 1000) and/or a liquid. For example, the second phase can decrease the power or energy density such that in the second phase the energy delivered is below the threshold for skin ablation but is above the threshold for ablation of the substance. For example, the energy density of the second phase can range from about 1% to about 90% of the energy density used in the first phase. In the second phase, the energy delivered pushes (e.g., forces) at least a portion of the substance 2300 into one or more of the plurality of micro-holes 1200.

In one embodiment, in the first phase, energy is delivered that ablates the plurality of micro-holes 1200 in the skin tissue 1000 (and causes at least some of the substance 2300 to be pushed into one or more of the micro-holes 1200) and in the second phase energy is delivered that ablates the substance 2300 (but does not ablate the skin tissue 1000) thereby enabling the substance 2300 (or an additional quantity of the substance 2300) to be pushed into the micro-hole 1200. In some embodiments, the first phase that ablates the micro-hole 1200 is created by one laser and the second phase of energy that ablates the substance 2300 is created by an energy source different from the laser used in the first phase (e.g., a different laser, a lamp, and/or a source of ultrasound energy). In other embodiments, the same laser is employed in the first phase and in the second phase; however, the laser employs one wavelength range in the first phase and another wavelength range in the second phase. For example, the ranges can include one or more wavelength(s) in a range from about 1.8 microns to about 11 microns, from about 1800 nm to about 3500 nm, or from about 180 nm to about 350 nm. FIG. 6E shows that the targeted volume of the substance 2300 is delivered to the micro-holes as a result of two phases of energy delivery (e.g., two separate pulses each of a plurality of micro-beams one in a wavelength range that ablates the tissue 1000 and the other in a wavelength range that ablates the substance 2300 thereby heating the substance 2300 up to its boiling point to create additional gas in the substance 2300).

FIGS. 7A-7D show embodiments of treating tissue with laser injection that employ an aligner 4000 to create ablated micro-holes and/or fill ablated micro-holes with a substance 2300. Suitable aligners 4000 fix and/or mark and/or frame the position of the surface 1100 of the skin tissue 1000 to be treated. The aligner 4000 allows the energy delivery source (e.g., the laser 1300) to return to the same position after it is removed. Thus, the aligner 4000 enables assured repeating of a location of skin tissue 1100 for treatment, e.g., with a plurality of microbeams. More specifically, referring now to FIG. 7A, an aligner 4000 is positioned adjacent to positions marked (Z) on the surface 1100 of skin tissue 1000. The energy source, e.g., the laser 1300, is positioned within the aligner 4000 and delivers a plurality of ablative micro beams through the surface 1100 of the skin tissue 1000. Optionally, the laser 1300 is removed from the aligner and remnants of ablated tissue particles can be wiped from the surface 1100 of the skin tissue 1000. Referring now to FIG. 7B, in one embodiment, a container component 2000A having an internal chamber filled with a substance 2300 is placed on the surface 1100 of the skin tissue 1000 in the region of position Z. The laser 1300 is positioned within the aligner 4000 and delivers a plurality of micro beams through the window 2100A and into the substance 2300. The plurality of micro beam delivered through the substance 2300 ablate and/or evaporate the substance 2300, thereby creating gas that increases the pressure within the internal chamber 2500 of the container component 2000A. FIG. 7C shows that the pressure generated by the energy delivered to the substance 2300 pushes and/or forces at least a portion of the substance 2300 to be displaced into one or more of the plurality of micro-holes 1200. FIG. 7D shows that the substance 2300 delivered to the plurality of micro-holes 1200 becomes trapped within the skin tissue 1000.

The aligner 4000 can provide the capability of aligning the energy delivery device with a region of skin tissue 1000 such that when the energy delivery device is moved away from and returned to the region of skin tissue 1000 the aligner 4000 ensures that the energy delivery device is positioned in the correct position (e.g., the same fixed position) each time the energy delivery device is returned to the aligner 4000. A suitable aligner 4000 can have any of a number of configurations. In one embodiment, the aligner 4000 is a frame that is placed adjacent a region of a skin surface 1100, optionally the region of the skin surface 1100 is marked where it is to match with the aligner 4000. In another embodiment, the aligner 4000 has at least two parts, one part that remains adjacent the region of the skin tissue 1000 to be treated and another portion that is disposed on the energy delivery device 1300. In one embodiment, the first part that remains adjacent the region of the skin tissue 1000 to be treated is a male part and the second portion is a complementary female part. Alternatively the first part remains adjacent the skin tissue 1000 and is a female part and the second part is a complementary male part.

In another embodiment, referring to FIG. 8A, an aligner 4000 is employed to align the energy emitting device (e.g., a laser 1300) with the surface 1100 of the skin tissue 1000. The laser 1300 is positioned within the aligner 4000 and delivers a plurality of ablative micro beams through the surface 1100 of the skin tissue 1000 forming a plurality of micro-holes 1200. The laser 1300 remains positioned in the same region of the skin tissue 1000. The container component 2000A has an internal chamber at least partially filled with a substance 2300. Referring to FIG. 8B, a handle 4050 is used to pull the container component 2000A over the surface 1100 of the skin tissue 1000 into the region of the micro beams delivered by the laser 1300. The aligner 4000 is sized and shaped to enable movement of the container component 2000A over the plurality of micro-holes 1200 while the aligner 4000 and optionally the laser 1300 are maintained in position. Referring still to FIG. 8B the energy emitting device (e.g., a laser 1300, a lamp, an ultrasound device) delivers energy suitable to ablate (e.g., laser energy at a wavelength suitable for ablation) and/or evaporate the substance 2300 held within the container component 2000A but not an energy suitable to ablate the skin tissue 1000. For example, the wavelength can be in a range from about 1.8 microns to about 11 microns, from about 1800 nm to about 3500 nm, or from about 180 nm to about 350 nm. In one embodiment, the energy source (e.g., a laser 1300) delivers a plurality of micro beams. In another embodiment, the energy source (e.g., a laser 1300) delivers a wide beam or a wide pulse. For example, a wide beam can also be referred to at a flat beam and/or a non-fractional beam. Further, the wide pulse can have a pulse range from a picosecond to a few second or a wide pulse can be at least 10 milliseconds. Referring now to FIG. 8C the increase in pressure in the internal chamber 2500 caused by the ablation and/or the evaporation of the substance 2300 held within the container component 2000A can push at least a portion of the substance 2300 into one or more of the plurality of micro-holes 1200.

Optionally, referring to FIGS. 8A and 8B, the surface 1100 of the skin tissue 1000 can be wiped free from skin particle debris created by ablating the plurality of micro-holes 1200 prior to moving the container component 2000A to the region of skin tissue 1000 having the plurality of micro-holes 1200.

Suitable applications of tissue clearing may be for masking the appearance of tattoo particles in the skin. For example, the substance 2300 may be selected in shape, color, transparency or other characteristics to mask the appearance of tattoo particles that are desired to be masked. The tattoo particles to be masked may be at a depth above, at, or below the depth of one or more of the micro-holes 1200 being at least partially filled by the substance 2300. In one embodiment, the micro-holes 1200 are at a depth at or above the dermal epidermal junction thereby enabling temporary masking of a tattoo for, for example, a special event or a desired period of time. By masking at or above the dermal epidermal junction, it is expected that the tattoo particles being masked will be revealed in time based upon the cycle of epidermal growth of skin and/or sloughing of skin. Masking below the dermal epidermal junction may enable permanent or substantially permanent coverage of tattoo particles.

Referring now to all of the embodiments employing a container component for injection of a substance into tissue, in one embodiment, the container component can contain a suspension of TiO₂ particles having a particle size of 100 nm in PEG-300 having a concentration of 0.5 g TiO₂/ml of PEG-300 (e.g., polyethylene glycol having a molecular weight of 300) within its internal chamber. The internal chamber of the container component can be filled with the suspension of TiO₂ particles two or more times during the method of injection of the substance into the micro-hole(s) (e.g., via laser injection and/or via ultrasound assisted injection). Refilling the internal chamber during the injection process can help to ensure that the micro-hole(s) are filled at the desired level.

Prior techniques to introduce substances into the tissue include techniques that relied on passive diffusion of the substance, which required a prolonged application time. In such techniques, the stratum corneum was disrupted though one or more of mechanical action, thermal action (heat or freezing), and/or acoustic/ultrasonic action followed by topical application of the substance, often with occlusion. Passive diffusion of the substance after topical application of the compound was not satisfactory for reasons including the prolonged application time.

Local enhancement of skin barrier permeability was believed to increase the efficacy of the insertion of topical compounds and to reduce the time-to-effect insertion of topical compounds that alter the optics of the skin. Challenges associated with local enhancement of skin barrier permeability include the need for controlled and reproducible methods that alter stratum corneum permeability that are reversible in order to preserve the long-term integrity of the skin barrier.

In order to ensure compound intake, a pressure gradient must be created in the skin barrier. In addition, there is a need to overcome the “flushing” action of blood flow, which can reduce the local concentration of the compound in dermis.

The laser injection approach disclosed herein includes the use of an ablative fractional laser that creates deep channels that assure penetration through and below the stratum corneum with a well-controlled, predictable pattern.

### Experiment

A study was conducted to deliver particles to skin tissue to mask a tattoo. More specifically, the intended application was to mask a tattoo by creating an additional scattering layer on top of the tattoo. In vivo tests were conducted with human tattoo models.

The study employed the StarLux 500 with the Lux2940 hand piece using the “Groove” optic and the Lux240 handpiece having a fractional tip with a pitch of 500 μm (750 μm on the skin). TiO₂ particles were employed to mask black tattoo ink comprising, for example, Carbon and/or Iron Oxide particles. The TiO₂ particles were inserted into the skin tissue and the success of masking the tattoo ink was determined visually and via Optical Coherent Tomography (OCT). A Dynatron-125 ultrasonic device was employed to aid in the injection of the substance into the micro-holes created in the skin tissue by the StarLux Lux 2940 system with the “Groove” optic and with the 500 μm optic.

This study was conducted in a fashion similar to the description associated with FIGS. 3A-3D. First, the skin tissue was treated by laser injection with the StarLux 2940 hand piece using the Groove optic and using the 500 μm (750 μm on the skin) optic. The StarLux 500 base unit was operated at parameters to provide energy per micro beam of 58 mJ at a pulse direction of about 135 μs. One or more ablated micro-holes were formed in the tissue at the wavelength of 2940 nm (a wavelength range suitable for ablation), similar to FIG. 3A. The 2940 handpiece was used to perforate skin by delivering at least one pulse with an energy level of 1.2 J.

A container component was positioned on the surface of the region of the skin tissue containing the previously ablated micro-hole(s). The container component provided an occlusive cover that was at least substantially sealed to the skin, similar to FIG. 3B. A Dynatron-125 ultrasonic device generated energy (delivering ultrasound or shock waves) through the window of the container component to reach the substance, a suspension of TiO₂ particles (having a 100 nm particle size) suspended in PEG.

Without being bound to any single theory, it is believed that the ultrasound energy that was transmitted through the container component window generated pressure for forcing at least a portion of the substance (e.g., the TiO₂ suspension) into the micro-holes. It is believed that the energy transmitted through the window converted at least a portion of the TiO₂ suspension from a suspension into the form of a gas and/or a liquid, which pushed the substance into the available space in the micro-holes, see FIGS. 3C and 3D.

The TiO₂ particles (having a 100 nm particle size) were delivered into human skin with the assistance of Ultrasound applied for 1 minute. The container component was removed from the surface of the skin tissue.

Three days after delivery of the TiO₂ particles to the skin tissue with the aid of ultrasound, the TiO₂ particles were visible in the skin via OCT. The presence of TiO₂ particles (masking an underlying tattoo) were visible in human skin up to 28 days after ultrasound assisted particle delivery. Thus, ultrasound may be used for accelerated particle delivery into the skin. The delivered particles can stay within the skin in vivo for at least several weeks.

While only certain embodiments have been described, it will be understood by those skilled in the art that various changes in form and details may be made therein without departing from the spirit and scope as defined by the appended claims. Those skilled in the art will recognize, or be able to ascertain using no more than routine experimentation, many equivalents to the specific embodiments described specifically herein. Such equivalents are intended to be encompassed in the scope of the appended claims.

The patent, scientific and medical publications referred to herein establish knowledge that was available to those of ordinary skill in the art. The entire disclosures of the issued U.S. patents, published and pending patent applications, and other references cited herein are hereby incorporated by reference.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent or later-developed techniques which would be apparent to one of skill in the art. In addition, in order to more clearly and concisely describe the claimed subject matter, the following definitions are provided for certain terms which are used in the specification and appended claims.

As used herein, the recitation of a numerical range for a variable is intended to convey that the embodiments may be practiced using any of the values within that range, including the bounds of the range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values ≧0 and ≦2 if the variable is inherently continuous. Finally, the variable can take multiple values in the range, including any sub-range of values within the cited range.

As used herein, unless specifically indicated otherwise, the word “or” is used in the inclusive sense of “and/or” and not the exclusive sense of “either/or.”

What is claimed is:

1. A method of driving a substance into a subject's skin, comprising:
placing a substance in contact with or in proximity to a portion of the skin;
applying energy to said skin portion so as to generate a plurality of micro-holes therein; and
applying energy to at least a portion of said substance to generate pressure for forcing at least a portion of said substance into one or more of the plurality of micro-holes.

2. The method of claim 1, wherein at least a portion of said substance changes its phase into a gaseous phase.

3. The method of claim 1, wherein at least a portion of said substance changes its phase into a liquid phase.

4. The method of claim 1, wherein said substance is disposed in a container having a surface adapted for contact with the skin.

5. The method of claim 4, wherein said surface is frangible and perforates in response to applying energy.

6. The method of claim 4, wherein said container is maintained in contact with the skin through the application of the energy to the skin and the substance.

7. The method of claim 1, wherein said substance alters the optics of the skin.

8. The method of claim 7, wherein said substance optically clears at least a portion of the skin appearance.

9. The method of claim 7, wherein said substance lightens at least a portion of the skin appearance.

10. The method of claim 7, wherein said substance protects at least a portion of the skin from UV-light.

11. The method of claim 7 wherein the substance within said micro-holes changes over time such that the optics of the skin returns to the unaltered optical appearance.

12. The method of claim 1, wherein the plurality of micro-holes have a depth at or above the dermal epidermal junction and the substance forced into the micro-holes are at a depth at or above the dermal epidermal junction.

13. The method of claim 1, wherein the plurality of micro-holes have a depth below the dermal epidermal junction and the substance forced into the micro-holes are at a depth below the dermal epidermal junction.

14. The method of claim 1, wherein the substance is disposed in a container, the container provides a seal with the skin when the container is in contact with the skin.

15. A method of driving a substance into a subject's skin, comprising:
placing a container housing in contact with a portion of the skin, said container housing defining a compartment containing a substance therein, the container housing configured to seal the compartment between the skin and the container housing when the compartment is in contact with the skin; and
applying ablative energy through at least a portion of the container housing thereby ablating said skin portion and so as to generate a plurality of micro-holes and such that a pressure within the compartment increases and drives at least a portion of said substance into said micro-holes with said increased pressure.

16. The method of claim 15, wherein at least a portion of said substance driven into said micro-holes is in a gaseous phase.

17. The method of claim 15, wherein at least a portion of said substance driven into said micro-holes is in a liquid phase.

18. The method of claim 15, wherein the container housing has a frangible surface that perforates in response to ablation.

19. The method of claim 15, wherein said container housing is maintained in contact with the skin through the application of the ablative energy to the skin and the substance.

20. The method of claim 15, wherein said substance alters the optics of the skin.

21. The method of claim 20, wherein said substance optically clears at least a portion of the skin appearance.

22. The method of claim 20, wherein said substance lightens at least a portion of the skin appearance.

23. The method of claim 20, wherein said substance protects at least a portion of the skin from UV-light.

24. The method of claim 1, wherein the plurality of micro-holes have a depth at or above the dermal epidermal junction and the substance forced into the micro-holes are at a depth at or above the dermal epidermal junction.

25. The method of claim 1, wherein the plurality of micro-holes have a depth below the dermal epidermal junction and the substance forced into the micro-holes are at a depth below the dermal epidermal junction.

26. A container component for driving a substance into tissue, the component comprising:
a compartment;
a window, at least a portion of the window is optically transparent to laser energy, the window reflects at least a portion of the sonic energy created when the laser energy is applied to tissue and the window prevents escape of the reflected acoustic sonic energy from the compartment; and
a wall, wherein at least a portion of the wall can form a seal with a tissue surface.

27. The container of claim 26 further comprising an orifice through which a substance is inserted into the compartment.

28. The container of claim 26, wherein the window comprises sapphire.
